(19) United States
(12) Patent Application Publication
(10) Pub. No.: US 2017/0266061 A1
(43) Pub. Date: Sep. 21, 2017

(54) MALE SANITARY SHIELD

(71) Applicants: Salvatore J. AIELLO, Naples, FL (US); Casimer J. Aiello, Naples, FL (US)

(72) Inventors: Salvatore J. AIELLO, Naples, FL (US); Casimer J. Aiello, Naples, FL (US)

(21) Appl. No.: 15/070,949

(22) Filed: Mar. 15, 2016

(51) Int. Cl. A61F 13/471; A61F 13/56

(52) U.S. Cl. CPC A61F 13/471; A61F 13/5611; A61F 2013/4708

(57) ABSTRACT

A male urine absorbent underclothing provides protection from small drops of urine due to enlarged prostate or water pills or the like. The male incontinence underclothing is preferably made of doubled Swiffer floor cleaner sheet and worn inside an undergarment worn by a user.

### CROSS-REFERENCE TO RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION-BY-REFERENCE OF MATERIALS SUBMITTED ON A COMPACT DISC

Not applicable.

### BACKGROUND OF THE INVENTION

1. Field of the Invention

The present invention relates to an absorbent device for male hygiene and protection. The invention, particularly relates to a urinary absorbent device to provide protection from small drops of urine due to enlarged prostate or water pills or the like of aging men.

2. Description of Related Art Including Information Disclosed Under 37 CFR 1.97 and 37 CFR 1.98.

Generally, incontinence is the inability of a person to control their bladder or bowel functions. More particularly, there is a serious problem of male penile urinary incontinence. For many male adults, young and old, urinary incontinence is a very sensitive yet serious problem. Male urinary incontinence may be caused by a variety of factors. It can occur, in varying degrees, for example, men who have undergone a prostatectomy (surgical removal of the prostate) or other urinary tract surgeries, such as a cystectomy (surgical removal of the bladder), in diabetics with nerve damage, and in men who have had a stroke or spinal cord injury or who suffer from Parkinson's disease or multiple sclerosis, as well as diseases such as priapism and Pyronie's. Aging is also a leading cause of male incontinence. Other causes include loss of control from such actions as coughing, sneezing, and lifting, and an abnormal and overwhelming urge to urinate caused, for example, by various bladder conditions such as exposure to radiation therapy. Finally, men who are handicapped or otherwise medically compromised may have problems with incontinence. Urinary incontinence can be more than an inconvenience and an embarrassment to men, it can cause discomfort from being in wet or damp clothing as well as genital itching or a rash or infection caused by contact with discharged urine.

For males who may not have elective surgery possibly due to health complications or old age, and for those males where the urinary incontinence cannot be surgically corrected, there are very few options. If surgery is not an option for an adult incontinent, then there only remains the use of adult diapers to soak up the discharging urine. However, diapers are very annoying, costly, unsightly and obtrusive. For an active person, diapers are a nuisance. For any adult, having to wear a diaper can be unpleasant and embarrassing.

Diapers are also very undesirable because they are unsanitary. Despite significant research and development, diapers still allow the excreted fluid to remain in contact with the skin until a new diaper is applied. However, this is a serious problem for an incontinent person who is continuously excreting small amounts of urine. It would be necessary to continuously change the diaper to avoid rashes and skin irritations completely.

Several differing techniques have been developed to either correct or ameliorate the symptoms resulting from penile incontinence. A number of different types of urine absorbing devices are present in prior art. For example, see: U.S. patent application Ser. No. 11/207,372 to Barbara; U.S. patent application Ser. No. 13/007,478 to Alexander; U.S. Pat. No. 5,300,052 to Yoshinori; U.S. Pat. No. 5,618,279 to Edward; U.S. Pat. No. 5,702,381 to Alan; U.S. Pat. No. 5,716,350 to Richard; U.S. Pat. No. 5,984,910 to Joseph; U.S. Pat. No. 6,817,992 to Sassak et al; U.S. Pat. No. 8,250,677 to Nicolosi; U.S. Pat. No. 8,277,426 to Wilcox et al.

Prior art, U.S. Pat. No. 8,277,426 discloses a male urinary incontinence device that helps prevent soiling of clothing with discharged urine, provide comfort to the male genitalia, allow sufficient freedom of movement to permit normal daily activities, and help separate the genitalia from discharged urine, thus providing a more natural feel includes a body. The male incontinence device including a body having a shell, and, the shell forming a cavity adapted to hold male genitalia. An absorbent, suitable for absorbing urine, is at least partially disposed within the cavity and a moisture barrier is positioned adjacent the body to help protect clothing from urine soiling. A liquid-permeable liner is attached to the body in a spaced-apart relation to the absorbent. To create a hammock-like effect, the liquid-permeable liner may be attached at the perimeter of the body.

Another prior art, U.S. Pat. No. 5,984,910 discloses an urinary incontinence device for use by a male that includes an undergarment worn closely to the body and including an opening or hole through which the penis passes and is kept upright and close to the body by a flap across the opener in the undergarment. The flap may include an occlusion device integrally incorporated therein and aligned with the opening to be positioned between the base of the shaft of the penis and the scrotum to occlude the urethral passage of the penis. The occlusion device is designed to apply sufficient pressure to occlude the urethral passage without cutting off circulation of blood within the penis through the dorsal vein. The flap design maintains and retains an optimum position of the penis and occlusion of the urethral passage to prevent urine discharge in a form fitting undergarment that allows significant freedom of movement by the wearer.

Thus, there is a need for a male urinating absorbent underclothing that is not only effective, but that fits the male genital anatomy comfortably, allows sufficient freedom of movement to permit normal daily activities, may be worn discretely, and that helps the genitals from becoming too warm and helps prevent the genitals from coming into contact with discharged urine.

### BRIEF SUMMARY OF THE INVENTION

In the view of the foregoing disadvantages inherent in the known types of absorbent underclothing now present in the prior art, the present invention provides an improved absorbent underclothing. As such, the general purpose of the present invention, which will be described subsequently in greater detail, is to provide a new and improved male urine absorbent underclothing which has all the advantages of the prior art and none of the disadvantages.

An object of the invention is to provide a male urine absorbent underclothing that provides protection from small drops of urine due to enlarged prostate or water pills or the like.

It is another object of the present invention to provide a male absorbent underclothing that is made of doubled Swiffer floor cleaner sheet.

It is another object of the present invention to provide a male absorbent underclothing worn inside an undergarment worn by a user.

It is another object of the present invention to provide an adhesive band attached to the absorbent pad to hold it in place.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE SEVERAL VIEWS OF THE DRAWINGS

The invention will be better understood and objects other than those set forth above will become apparent when consideration is given to the following detailed description thereof. Such description makes reference to the annexed drawings wherein:

FIG. 1 shows the top view of the absorbent pad placed outside the undergarment.

FIG. 2 shows the preferred embodiment of the absorbent pad.

### DETAILED DESCRIPTION OF THE INVENTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description is, therefore, not to be taken in a limiting sense, and the scope of the present invention is defined by the appended claims and their equivalents.

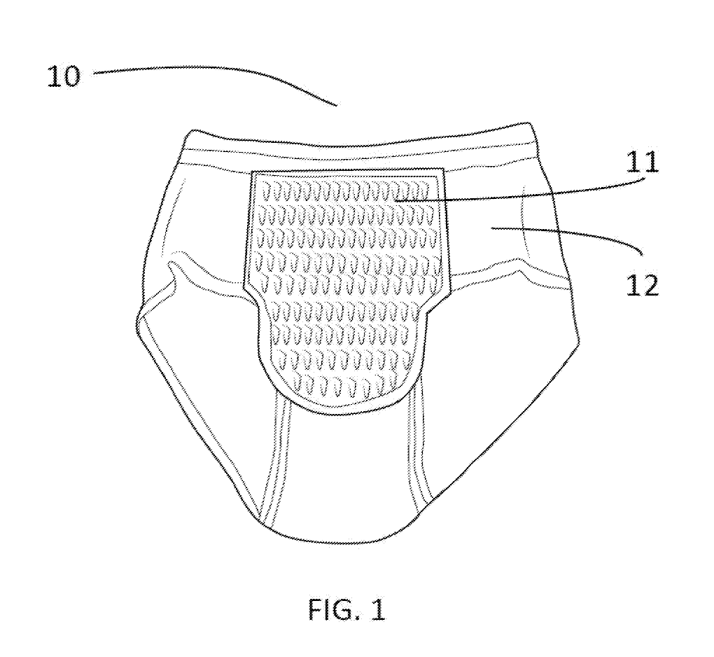

In one embodiment, shown in FIG. 1, a male urine absorbent underclothing 10 includes an absorbent pad 11 that is worn inside the undergarment of an individual. The figure shows the absorbent pad placed over and outside the front section of the undergarment 12 of a male. The absorbent pad is made up of double Swiffer floor cleaner sheet. This material is soft to the skin and undetectable by the wearer.

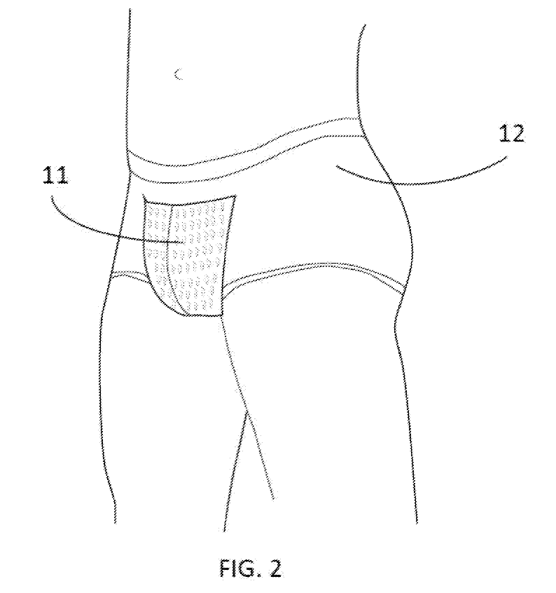

In a preferred embodiment, shown in FIG. 2, the absorbent pad 11 is placed between the inner side of the front section of the underwear 12 or undergarment or like and genitals of the user or male. The absorbent pad absorbs the urine drops and in effect leaves the undergarment dry.

The sanitary underclothing of the current invention provides protection from small drops of urine due to enlarged prostate or water pills or the like. The protection avoids embarrassing spots and odor on user slacks or shorts. The sanitary underclothing is a flat surface product. It allows the user to feel confident during the day or the evening. It is a one use product and is flushable.

It is to be understood that the above description is intended to be illustrative, and not restrictive. For example, the above-discussed embodiments may be used in combination with each other. Many other embodiments will be apparent to those of skill in the art upon reviewing the above description.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention.

I claim:

1. A male sanitary underclothing, comprising:
an absorbent pad; and
an adhesive band attached to said absorbent pad.

2. The male sanitary underclothing according to claim 1, wherein said absorbent pad is placed inside an undergarment worn by a user.

3. The male sanitary underclothing according to claim 2, wherein said absorbent pad is placed in between an inner side of a front section of said undergarment and genitals of said user.

4. The male sanitary underclothing according to claim 1, wherein said adhesive band holds said absorbent pad in place.

5. The male sanitary underclothing according to claim 1, wherein said absorbent pad is made of doubled Swiffer floor cleaner sheet.
